(19) United States
(12) Patent Application Publication
(10) Pub. No.: US 2026/0363780 A1
(43) Pub. Date: Nov. 19, 2026

(54) PLANNING SYSTEMS AND METHODS FOR DYNAMIZATION USING EXTERNAL FIXATION SYSTEMS

(71) Applicant: ARTHREX, INC., Naples, FL (US)

(72) Inventor: Scott Smyth, Naples, FL (US)

(21) Appl. No.: 19/676,949

(22) Filed: May 14, 2026

Related U.S. Application Data
(60) Provisional application No. 63/807,110, filed on May 16, 2025.
(60) Provisional application No. 63/901,913, filed on Oct. 20, 2025.
(60) Provisional application No. 64/048,205, filed on Apr. 24, 2026.

(51) Int. Cl. A61B 34/10; A61B 17/66

(52) U.S. Cl. CPC A61B 34/10; A61B 17/66; A61B 2034/104; A61B 2034/105

(57) ABSTRACT

This disclosure relates to planning systems and methods for performing orthopaedic surgeries using external fixation systems, which may stabilize complex fractures and/or correcting limb deformities. The external fixation system may include one or more struts that connect between a pair of supports. Adjustments for the struts may be determined to establish a direction of collapse of the external fixation system for providing dynamization.

### CROSS-REFERENCE TO RELATED APPLICATIONS

This application claims the benefit of U.S. Provisional Application No. 63/807,110, which was filed on May 16, 2025.

The application further claims the benefit of U.S. Provisional Application No. 63/901,913, which was filed on Oct. 20, 2025.

This application further claims the benefit of U.S. Provisional Application No. 64/048,205, which was filed on Apr. 24, 2026.

The disclosures of each of the above applications are incorporated herein by reference in their entirety.

### BACKGROUND

This disclosure relates to external fixation systems, including planning and implementing dynamization during bone consolidation.

External fixation systems are surgical devices that may be used to stabilize complex fractures and/or correct limb deformities. Some external fixation systems utilize rings or platforms that are connected together by rods and/or struts.

### SUMMARY

This disclosure is directed to planning systems and methods performing orthopaedic surgeries including dynamization of bones with external fixation systems. The external fixation systems may be used to perform orthopaedic surgeries for stabilizing complex fractures and/or correcting limb deformities. The external fixation system may include one or more struts. One or more adjustments to the struts may be determined for dynamization.

A system for planning an orthopaedic procedure may include one or more processors coupled to memory. The one or more processors may be operable to collectively execute a planning environment. The planning environment may be operable to establish a resection plane along a bone model which may be associated with a bone of a patient. The planning environment may be operable to determine a displacement vector which may be associated with compression of the bone adjacent to a resection associated with the resection plane during consolidation. The displacement vector may be substantially normal to the resection plane. The planning environment may be operable to place a fixation model relative to the bone model. The fixation model may be associated with an external fixation device including a plurality of struts interconnecting first and second supports securable to the bone. The struts may be adjustable to collectively establish a direction of collapse of the external fixation device. The planning environment may be operable to determine a set of strut vectors associated with the respective struts. The planning environment may be operable to establish a dynamization schedule including adjustments that may vary an amount of permitted dynamization travel of one or more of the struts based on the strut vectors such that the direction of collapse may be substantially parallel to the displacement vector.

A system for planning an orthopaedic procedure may include one or more processors coupled to memory. The one or more processors may be operable to collectively execute a planning environment. The planning environment may be operable to establish a resection plane along a three-dimensional bone model which may be associated with a bone of a patient. First and second portions of the bone model may be on opposite sides of the resection plane. The planning environment may be operable to determine a displacement vector associated with compression of the bone during consolidation. The displacement vector may be substantially normal to the resection plane. The planning environment may be operable to place a three-dimensional fixation model about a periphery of the bone model. The fixation model may include a plurality of struts that may be distributed about an axis and may interconnect first and second supports. The first and second supports may have positions fixed relative to the respective first and second portions of the bone model. The struts may be adjustable to collectively establish a direction of collapse of the fixation model. The planning environment may be operable to determine a set of strut unit vectors associated with the respective struts. The planning environment may be operable to determine adjustments that may vary an amount of permitted dynamization travel of the respective struts based on the strut unit vectors such that the direction of collapse may be substantially parallel to the displacement vector in response to the adjustments.

A method of performing an orthopaedic procedure may include establishing a resection plane along a bone model which may be associated with a bone of a patient. The method may include determining a displacement vector associated with compression of the bone during consolidation. The compression may be along a resection associated with the resection plane. The displacement vector may be substantially normal to the resection plane. The method may include placing a fixation model relative to the bone model. The fixation model may be associated with an external fixation device and may include a plurality of struts interconnecting first and second supports securable to the bone. The struts may collectively establish a direction of collapse of the external fixation device. The method may include determining a set of strut unit vectors associated with the respective struts. The method may include establishing a dynamization schedule including adjustments that may vary an amount of permitted dynamization travel of the respective struts based on the strut unit vectors such that the direction of collapse may be substantially parallel to the displacement vector.

The present disclosure may include any one or more of the individual features disclosed above and/or below alone or in any combination thereof.

The various features and advantages of this disclosure will become apparent to those skilled in the art from the following detailed description. The drawings that accompany the detailed description can be briefly described as follows.

### BRIEF DESCRIPTION OF THE DRAWINGS

FIG. 1 discloses a perspective view of an external fixation system including struts between supports according to an implementation.

FIG. 2 discloses the external fixation system of FIG. 1 arranged relative to a bone.

FIG. 3 discloses an implementation of a strut including a dynamization mechanism.

FIG. 4 discloses the dynamization mechanism of the strut of FIG. 3 in a locked position.

FIG. 5 discloses a section view of the dynamization mechanism taken along line 5-5 of FIG. 4.

FIG. 6A discloses a perspective view of the dynamization mechanism of the strut of FIG. 3 in the locked position.

FIG. 6B discloses a perspective view of the dynamization mechanism of the strut of FIG. 3 in an unlocked position.

FIG. 7 discloses a perspective view of the dynamization mechanism of the strut of FIG. 3 in the unlocked position and the strut in a collapsed state.

FIG. 8 discloses a planning system according to an implementation.

FIG. 9 discloses a bone model in a user interface of the planning system of FIG. 8.

FIG. 10 discloses a device model relative to the bone model in the user interface of the planning system of FIG. 8.

FIG. 11 discloses the device model and strut vectors associated with the struts of the device model of FIG. 10.

FIG. 12 discloses a method of planning and implementing an orthopaedic procedure.

Like reference numbers and designations in the various drawings indicate like elements.

### DETAILED DESCRIPTION

This disclosure is directed to external fixation systems (e.g., devices or frames) for performing orthopaedic surgeries for stabilizing complex fractures and/or correcting limb deformities. The external fixation systems may include one or more struts that connect between a pair of supports. The struts may be adjustable to provide dynamization to the bone.

The disclosed systems and methods may be utilized with various external fixations systems. An external fixation system may be used to stabilize complex fractures and/or correct limb deformities. The external fixation system may be configured as a hexapod-type dynamic external fixation system, an Ilizarov-type static external fixation system, or any other type of external fixation system. The fixation system may be arranged about and secured to one or more bones of a human musculoskeletal system. The bone(s) may be part of any limb or joint of the human musculoskeletal system, including but not limited to that of the leg, hand, wrist, forearm, foot, or ankle.

The external fixation system may include a plurality of supports (e.g., platforms or plates). Each support of the system may be either a full ring or a partial ring and may be arranged to circumferentially extend at least partially around the bone(s). The supports may be fixedly attached to bone.

Adjacent pairs of supports of the external fixation system may be connected together by one or more length-adjustable struts. Each support may include openings for receiving portions of the struts. The struts may connect between fixation points of the struts to allow for precise adjustment of the system relative to the bone(s). The struts may be adjustable to establish a compressive force along an interface between adjacent portions of the bone, including adjacent resection surfaces which may be brought into abutment to establish a correction.

The disclosed systems and methods may be utilized in the treatment of bone deformities. The treatment may include a deformity correction phase. The deformed bone may be resected (e.g., cut) to establish two or more bone portions (e.g., segments). The bone portions may be translated and/or angled relative to each other (e.g., 1-2 mm per day) until the correction is completed. Following deformity correction or distraction, the resected surfaces of the bone portions (e.g., segments) may either be brought into abutment (e.g., acute correction osteotomy) or separated by a regenerate zone (e.g., distraction osteogenesis). In both implementations, consolidation may occur as the bone tissue bridges (e.g., fills) the resection or regenerate region between the bone segments. Dynamization may then be performed to stimulate healing, either by promoting axial compression across abutted resection surfaces or by progressively loading the regenerate zone to encourage callus maturation, mineralization, and remodeling. The external fixation device may be fixed to the bone portions and may take an entirety of the load (e.g., bodyweight) during the correction phase.

Once the correction phase is complete, the bone may consolidate which may include fusing of the resected surfaces of the bone portions. Consolidation may be achieved using dynamization. Dynamization may be used in orthopaedic fracture and post-deformity correction osteotomy management. Dynamization may be performed by causing a controlled destabilizing (e.g., reduction in a rigidity) of an external fixation frame to stimulate bone healing during the final phases of treatment (e.g., bone consolidation). Dynamization may stimulate healing of the corrected bone. Load sharing may be gradually transferred from the external fixation device to the corrected bone. An (e.g., overall) controlled collapse of the external fixation device may be utilized to establish the load transfer. Portions of the struts may be configured to freely travel (e.g., float) relative to each other to provide the controlled collapse. The portions may freely travel relative to each other in an axial direction, which may be with respect to a longitudinal axis of the strut. The controlled collapse of the external fixation device may be established by an amount of permitted dynamization travel (e.g., axial displacement) of the struts. The dynamization travel may include a collapse and/or expansion of the struts. The struts may be configured to set the amount of permitted dynamization travel, which may be performed using any of the techniques disclosed herein. All struts may collapse, or some struts may collapse while other struts may expand, to provide the overall controlled collapse of the external fixation device. A maximum amount of free travel (e.g., collapse and/or expansion) of the struts may be limited by the permitted dynamization travel. The gradual transfer may promote healing of the bone along the resection. The transfer may be completed over several days, weeks or months. Dynamization may allow for controlled compression and micromotion at the resection site to promote bone union. By allowing carefully directed micro-movements at the bone interface, dynamization may stimulate the bone and may encourage natural biological healing processes such as callus maturation, mineralization, and remodeling. A dynamization (e.g., clinical prescription) schedule may be established and may be incorporated into the surgical plan for treating the patient. The struts may be adjusted based on the dynamization schedule.

Prior techniques may utilize a random approach to adjusting the struts of an external fixation device for providing dynamization, which may include randomly loosening bolts on different sides of the fixation device. This may introduce torsion or shear between the resection surfaces.

For dynamization to be clinically successful, stimulation at the fracture or osteotomy site may need to occur substantially purely perpendicular to the osteotomy plane (e.g., along the normal vector) to provide pure axial compression at the healing surface. If the destabilizing of the fixation device introduces shear forces (e.g., sideways sliding motion) across the osteotomy or fracture, it may cause micro-instability, delayed union, non-union or malalignment, which may impede the patient's recovery.

The disclosed systems and methods may be utilized to provide a controlled dynamization travel (e.g., collapse and/or expansion) of the struts and a controlled collapse of the associated fixation device to achieve dynamization, which may be performed in a reproduceable manner. The dynamization may cause micromotion between the resection surfaces (e.g., less than 2-3 mm). The disclosed techniques may be implemented to maintain axial compression between the resection surfaces during the dynamization but may reduce shear and torsion which may otherwise cause a malunion or non-union between the adjacent bone portions. Less than 3 mm (e.g., 1-2 mm) of micromotion may be achieved during consolidation of the corrected bone, which may promote bone regeneration.

The disclosed struts may include a dynamization mechanism (e.g., feature) that may be manipulated to establish a controlled collapse of the external fixation device. Dynamization may allow the strut to float (e.g., collapse and/or expand) along a strut (e.g., longitudinal) axis. The dynamization mechanism may be adjustable to vary an axial rigidity (e.g., stiffness) of the strut such that the strut may collapse (e.g., compress) and/or expand along the strut axis, which may occur in response to loading the strut and/or moving the strut automatically back and forth using one or more devices (e.g., motor or servo). The axial rigidity of the strut may be defined as its stiffness in response to axial (e.g., compressive) forces. The dynamization mechanism may be adjustable to permit a controlled collapse or expansion of the strut by an (e.g., preselected or permitted) amount of dynamization travel. The struts may be individually adjustable, including with different amounts, to provide dynamization. The adjustments may be associated with a per-strut dynamization travel (e.g., collapse and/or expansion) for achieving the planned dynamization amount. The adjustments may be continuously variable or may include discrete values.

The dynamization mechanism may include a spring (e.g., biasing) member, which may oppose a length change (e.g., collapse) of the strut. The spring member may cause the strut to expand in response to a lessening of the load. The dynamization mechanism may be adjustable to reduce a spring force of the spring member, which may include causing the spring member to elastically return to its original length and which may increase a transfer of load sharing from the external fixation device to the corrected bone.

The disclosed planning systems and methods may be utilized to determine one or more combinations of adjustments that may be incorporated into the dynamization schedule to achieve a controlled collapse of the fixation device. The struts may be adjustable to (e.g., individually and/or collectively) establish a direction of (e.g., controlled) collapse of the fixation device. Utilizing the techniques disclosed herein, the adjustments may be determined such that the direction of collapse may be substantially perpendicular to the osteotomy plane. Per-strut adjustments may be determined for collapsing the fixation device straight along the healing plane, which may reduce shear, torsion and a likelihood of compromising an integrity of the fixation device. By ensuring pure axial loading across the osteotomy during bone consolidation, the system may promote faster, stronger and more reliable bone healing, which may directly improve patient outcomes and reduce the risk of surgical revision.

The disclosed techniques may incorporate regression analysis, including a least-squares computation. The regression analysis may evaluate a surgeon-defined osteotomy (e.g., resection) plane and an arrangement of the struts to achieve the controlled collapse. The resection plane may be associated with various geometries, such as a planar or curved cut. Various techniques may be utilized to set a resection (e.g., osteotomy) plane. The resection plane may be established based on a set of points, a combination of points, and/or one or more lines in one or more (e.g., different) views. In implementations, the surgeon may select three points (e.g., two in an anterior-posterior view and one in a medial-lateral view). An osteotomy plane normal vector may be determined. Strut unit vectors may be determined based on a geometry of the fixation device. A target displacement value may be determined, which may be specified (e.g., prescribed) by the surgeon. The target displacement may be associated with compression along the osteotomy plane. The target displacement value may or may not correlate to the exact displacement of the bone at the osteotomy (e.g., regenerate) site. The target displacement value may be a measure of allowed (e.g., permitted) geometric displacement (e.g., motion). The regenerate tissue (e.g., callus) may have a stiffness that may change over time as consolidation progresses, which may absorb more of the permitted motion over time. A strut matrix may be established using the strut unit vectors. A target displacement vector may be determined based on the target displacement. A Gram matrix may be established based on the strut matrix. The Gram matrix may be inverted using various techniques, such as an adjugate/determinant method. A pseudo-motion vector may be solved. The dynamization (e.g., adjustment) values for each strut may be determined by projecting the pseudo-motion vector onto the strut unit vector, which may be incorporated into the dynamization schedule.

A system for planning an orthopaedic procedure may include one or more processors coupled to memory. The one or more processors may be operable to collectively execute a planning environment. The planning environment may be operable to establish a resection plane along a bone model which may be associated with a bone of a patient. The planning environment may be operable to determine a displacement vector which may be associated with compression of the bone adjacent to a resection associated with the resection plane during consolidation. The displacement vector may be substantially normal to the resection plane. The planning environment may be operable to place a fixation model relative to the bone model. The fixation model may be associated with an external fixation device including a plurality of struts interconnecting first and second supports securable to the bone. The struts may be adjustable to collectively establish a direction of collapse of the external fixation device. The planning environment may be operable to determine a set of strut vectors associated with the respective struts. The planning environment may be operable to establish a dynamization schedule including adjustments that may vary an amount of permitted dynamization travel of one or more of the struts based on the strut vectors such that the direction of collapse may be substantially parallel to the displacement vector.

In any implementations, the permitted dynamization travel may include a collapse and/or expansion of the respective one or more struts.

In any implementations, the planning environment may be operable to determine the adjustments based on regression. The regression may be based on the strut vectors and the displacement vector.

In any implementations, the regression may include a least-squares regression. The least-squares regression may include a strut matrix associated with the strut vectors. The least-squares regression may include a Gram matrix based on the strut matrix and a Gram matrix determinant. The Gram matrix determinant may be associated with a distribution of movement of the struts to collectively establish the direction of collapse.

In any implementations, the least-squares regression may include a pseudo-motion vector that may establish a set of best-fit translation coefficients. The pseudo-motion vector may be based on an inversion of the Gram matrix and the displacement vector. The least-squares regression may include a projection of the pseudo-motion vector onto the strut vectors that may establish dynamization amounts. The least-squares regression may include respective amounts of total dynamization displacement for the struts that may establish the adjustments in the dynamization schedule. The amounts of total dynamization displacements may be based on a magnitude of the displacement vector, a magnitude of the pseudo-motion vector and the dynamization amounts.

In any implementations, one or more of the adjustments may cause an increase in the amount of permitted dynamization travel of one or more of the respective struts.

In any implementations, one or more of the adjustments may cause a decrease in the amount of permitted dynamization travel of one or more of the respective struts.

In any implementations, the adjustments may include different amounts of permitted dynamization travel associated with the struts.

In any implementations, the strut may include a strut body. The strut may include a strut shaft that may be translatably received within an internal cavity of the strut body to set a length of the strut associated with the respective strut vector. The strut may include a dynamization mechanism that may be adjustable to vary the amount of permitted dynamization travel of the strut.

In any implementations, the dynamization mechanism may include a dynamization adjustment control that may be adapted to move relative to a dynamization shaft to either lock or unlock dynamization of the strut in respective locked and unlocked modes. A position of the dynamization adjustment control may be adjustable in the unlocked mode to vary the amount of permitted dynamization travel of the respective strut. The strut body may be moveable relative to the dynamization shaft in the unlocked mode but not in the locked mode.

In any implementations, the dynamization mechanism may include a spring member that may oppose axial movement of the strut body relative to the dynamization shaft in the unlocked mode.

In any implementations, the planning environment may be operable to display the fixation model relative to the bone model in a display window of a graphical user interface. The planning environment may be operable to establish the resection plane based on a set of resection points along the bone model.

In any implementations, the set of resection points may be set in response to user interaction with the user interface.

In any implementations, the resection plane may be associated with a resection that may divide the bone into a first portion attached to the first support and a second portion attached to the second support.

In any implementations, the external fixation system may include a hexapod configuration having six of the struts.

A system for planning an orthopaedic procedure may include one or more processors coupled to memory. The one or more processors may be operable to collectively execute a planning environment. The planning environment may be operable to establish a resection plane along a three-dimensional bone model which may be associated with a bone of a patient. First and second portions of the bone model may be on opposite sides of the resection plane. The planning environment may be operable to determine a displacement vector associated with compression of the bone during consolidation. The displacement vector may be substantially normal to the resection plane. The planning environment may be operable to place a three-dimensional fixation model about a periphery of the bone model. The fixation model may include a plurality of struts that may be distributed about an axis and may interconnect first and second supports. The first and second supports may have positions fixed relative to the respective first and second portions of the bone model. The struts may be adjustable to collectively establish a direction of collapse of the fixation model. The planning environment may be operable to determine a set of strut unit vectors associated with the respective struts. The planning environment may be operable to determine adjustments that may vary an amount of permitted dynamization travel of the respective struts based on the strut unit vectors such that the direction of collapse may be substantially parallel to the displacement vector in response to the adjustments.

In any implementations, the planning environment may be operable to determine an amount of total dynamization displacement of the respective struts to achieve a target displacement associated with the displacement vector based on regression. The regression may be based on the strut unit vectors and the displacement vector.

In any implementations, the adjustments may have different amounts of permitted dynamization travel associated with the struts.

A method of performing an orthopaedic procedure may include establishing a resection plane along a bone model which may be associated with a bone of a patient. The method may include determining a displacement vector associated with compression of the bone during consolidation. The compression may be along a resection associated with the resection plane. The displacement vector may be substantially normal to the resection plane. The method may include placing a fixation model relative to the bone model. The fixation model may be associated with an external fixation device and may include a plurality of struts interconnecting first and second supports securable to the bone. The struts may collectively establish a direction of collapse of the external fixation device. The method may include determining a set of strut unit vectors associated with the respective struts. The method may include establishing a dynamization schedule including adjustments that may vary an amount of permitted dynamization travel of the respective struts based on the strut unit vectors such that the direction of collapse may be substantially parallel to the displacement vector.

In any implementations, the step of establishing the dynamization schedule may include determining the adjustments based on regression. The regression may be based on the strut unit vectors and the displacement vector.

In any implementations, the step of performing the regression may include establishing a strut matrix associated with the strut unit vectors. Performing the regression may include establishing a Gram matrix based on the strut matrix and a Gram matrix determinant. The Gram matrix determinant may be associated with a distribution of movement of the struts to collectively establish the direction of collapse.

In any implementations, the step of performing the regression may include determining a pseudo-motion vector that may establish a set of best-fit translation coefficients. The pseudo-motion vector may be based on an inversion of the Gram matrix and the displacement vector. Performing the regression may include determining a projection of the pseudo-motion vector onto the strut unit vectors to determine the adjustments in the dynamization schedule.

In any implementations, the external fixation device may be secured to the bone. The method may include adjusting the struts of the external fixation device during consolidation of the bone according to the dynamization schedule.

In any implementations, the adjusting step may include moving a dynamization adjustment control of the strut based on an amount specified by one of the adjustments in the dynamization schedule to adjust the amount of permitted dynamization travel of the strut.

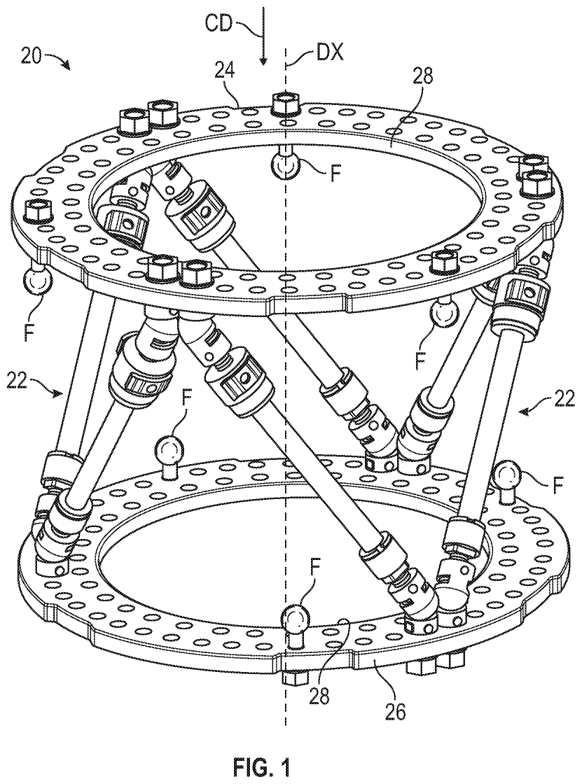

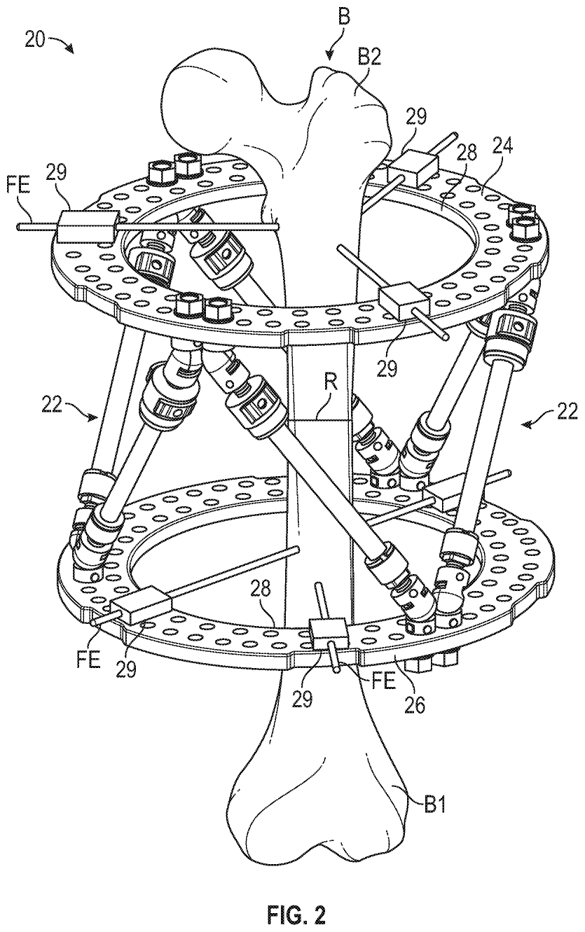

FIGS. 1-2 disclose an external fixation system (e.g., device) 20 according to an implementation. The fixation system 20 may be utilized in various orthopaedic procedures including correcting one or more deformed bones. The fixation system 20 may include on or more struts 22, which may interconnect first and second supports (e.g., platforms or plates) 24, 26. The struts 22 may be distributed about a device (e.g., longitudinal) axis DX (e.g., FIG. 1). The supports 24, 26 may be arranged along the device axis DX. The supports 24, 26 may have various geometries. In the implementation of FIGS. 1-2, the supports 24, 26 may have a ring-shaped geometry. The supports 24, 26 may have respective openings 28. In the implementation of FIG. 2, the openings 28 may be dimensioned to at least partially receive one or more bones of the patient anatomy. The bones may include long bones such as a femur, a tibia, ulna and/or radius. The supports 24, 26 may be fixedly attached or otherwise securable to portions of a bone. In implementations, the supports 24, 26 may include one or more guide structures 29 to place respective fixation elements FE in the bone. Various fixation elements may be utilized, such as Kirschner wires, pins and screws. The surgeon may cross-pin the bone to limit relative movement with the fixation system 20.

The fixation system 20 may include one or more fiducials (e.g., markers) F. The fiducials F may be fixedly attached or otherwise secured to the respective supports 24, 26. The fiducials F may have a (e.g., known or identifiable) geometry and may be used to determine a two-dimensional and/or three-dimensional orientation of the supports 24, 26 of the fixation system 20 relative to patient anatomy in one or more (e.g., fluoroscopic) images. The fiducials F may have various geometries, including a portion having a ball-shaped geometry. In implementations, three fiducials F may be securable to the support 24/26. Images of the supports 24, 26 and fiducials F in two different views may be utilized to determine a placement of the support 24/26 relative to the patient anatomy. The position of the struts 22 may be determined based on indexing the struts 22 relative to discrete positions along the supports 24, 26.

The struts 22 may be pivotably connected to the supports 24, 26. The strut 22 may be adjustable to vary a length of the strut 22 and/or a distance between the supports 24, 26. The struts 22 may be adjustable (e.g., shortened) to apply a compressive force along a resection of the bone. In the implementation of FIGS. 1-2, the fixation system 20 may include a hexapod configuration having a total of six of the struts 22. The struts 22 may be adjustable to correct multi-planar bone deformities in six degrees of freedom. The fixation system 20 may include fewer or more than six struts 22, such as only two or three struts 22.

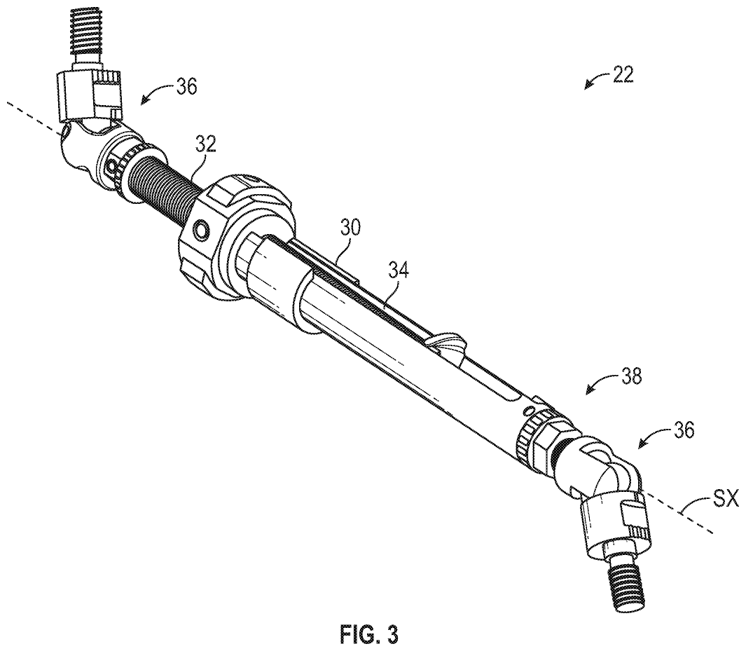

Referring to FIG. 3, with continuing reference to FIGS. 1-2, the strut 22 may include a strut body 30 and/or a strut shaft (e.g., rod assembly) 32. The strut body 30 and/or the rod assembly 32 may extend along a strut (e.g., longitudinal) axis SX. The strut 22 may be length-adjustable. The shaft 32 may be translatably received within an internal cavity 34 of the strut body 30 to set a length of the strut 22. The length may be defined with respect to the strut axis SX. The shaft 32 may include threading adapted to mate with threading along the strut body 30 to set the length of the strut 22. In the implementation of FIG. 2, bone portions (e.g., segments) B1, B2 of a bone B may abut along a resection R. The supports 24, 26 may be fixed to the respective bone portions B1, B2. The length of the strut 22 may be varied (e.g., reduced) to apply a compressive force between the adjacent bone portions B1, B2 of the resected bone B when the supports 24, 26 are fixed to the respective bone portions B1, B2.

The strut 22 may include one or more joint assemblies 36. The joint assemblies 36 may be adapted to pivotably couple the strut 22 to the supports 24, 26. In implementations, the joint assemblies 36 may include a ball joint configuration. The joint assemblies 36 may be adapted to permit relative rotation between the strut 22 and the supports 24, 26. The joint assembly 36 may be selectively locked to limit relative rotation between the strut 22 and supports 24, 26.

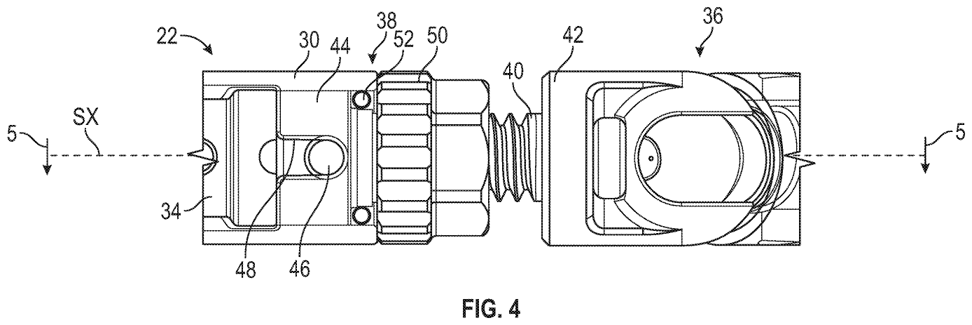

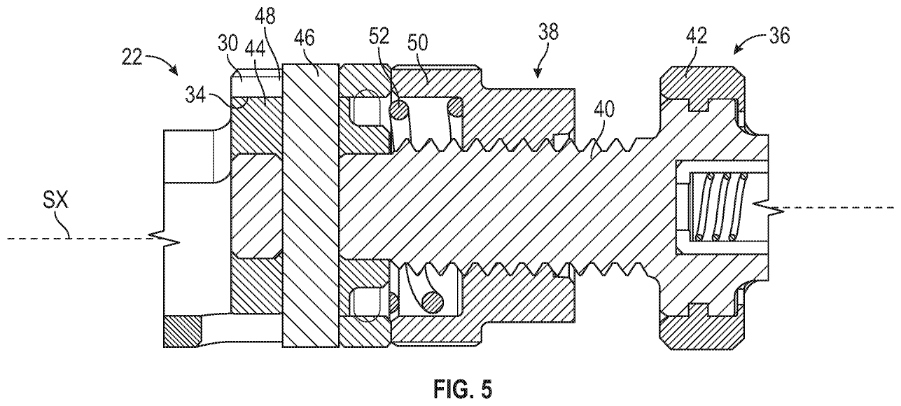

The struts 22 may be adjustable to (e.g., collectively) establish a direction of collapse CD of the external fixation device 20 (e.g., FIG. 2), which may establish dynamization during consolidation of a (e.g., corrected) bone. The direction of collapse CD may be substantially parallel to the device axis DX. The strut 22 may include a dynamization mechanism (e.g., feature) 38. The dynamization mechanism 38 may be utilized to achieve an (e.g., target or prescribed) dynamization. The dynamization mechanism 38 may be adjustable to vary (e.g., increase or reduce) an axial rigidity of the respective strut 22. The dynamization mechanism 38 may be adjustable to vary (e.g., reduce or increase) an amount of permitted dynamization travel (e.g., collapse and/or expansion) of the strut 22. The permitted dynamization travel may include movement in an axial direction relative to a strut axis SX of the strut 22 (e.g., FIGS. 4-5). The permitted dynamization travel may substantially exclude movement in a radial direction relative to the strut axis SX. The dynamization mechanism 38 may be adapted to cause a dimension (e.g., length) of the strut 22 to vary (e.g., reduce) when the bone may be loaded, including with a bodyweight of patient. The strut 22 may collapse by the permitted amount in response to the load. The permitted dynamization travel may differ from a length adjustment of the strut 22, which may be set to achieve correction of a bone deformity. The dynamization travel may occur subsequent to an end of correction (e.g., prescription) of the bone. The strut 22 may be configured such that the dynamization travel does not cause the strut 22 to increase in maximum length, which may be set by a length adjustment prior to configuring the strut 22 for dynamization.

The dynamization mechanism 38 may be adjustable using automated (e.g., robotic) and/or manual techniques. In implementations, the dynamization mechanism 38 may incorporate a servo to drive rotation and/or axial displacement of the strut 22.

Referring to FIGS. 4-5, with continuing reference to FIG. 3, the dynamization mechanism 38 may include a dynamization shaft 40. A first end of the dynamization shaft 40 may be fixedly attached or otherwise secured to a joint body 42 of the joint assembly 36. A second end of the dynamization shaft 40 may be coupled to a coupler 44, which may be securable to the strut body 30.

Various techniques may be utilized to secure the coupler 44 to the strut body 30. In implementations, the coupler 44 may be at least partially received within the internal cavity 34 of the strut body 30. The coupler 44 may be dimensioned to at least partially receive a retention (e.g., cross) pin 46. The retention pin 46 may be captured within a retention slot 48, which may be established within the strut body 30. The retention slot 48 may be dimensioned to limit (e.g., axial) movement of the retention pin 44 along the retention slot 48, which may limit (e.g., axial) relative movement between the strut body 30 and the joint body 42 of the joint assembly 36.

The dynamization mechanism 38 may include a dynamization adjustment control 50. In implementations, the adjustment control 50 may be a (e.g., rotatable) control knob, which may be turned to either lock or unlock the dynamization. The adjustment control 50 may include threading that may be adapted to engage threading along the dynamization shaft 40.

Various techniques may be used to cause the strut 22 to expand during dynamization. The dynamization feature 38 may include a spring member 52. The spring member 52 may be captured between the coupler 44 and adjustment control 50. The spring member 52 may include various spring configurations, such as a coil or wave spring. The spring member 52 may be utilized to substantially maintain an original (e.g., end of prescription) length of the strut 22 when unloaded. For the purposes of this disclosure, the term “substantially” means ±2 percent of the stated value or relationship unless otherwise indicated. The spring member 52 may be adapted to bias the strut body 30 in a direction away from an opposite end of strut 22 and/or adjustment control 50, which may be along the strut axis SX. The adjustment control 50 may be moveable to vary a compression of the spring member 52. In implementations, the adjustment control 50 may be rotatable about the strut axis SX to vary the compression of the spring member 52.

The adjustment control 50 may be movable (e.g., rotatable) to vary or otherwise set an amount of permitted strut dynamization travel. The adjustment control 50 may include continuously variable adjustments or may include discrete values. The adjustments may be associated with indicia (e.g., laser marks), which the surgeon or clinical user may evaluate to confirm implementation of a (e.g., prescribed) dynamization schedule.

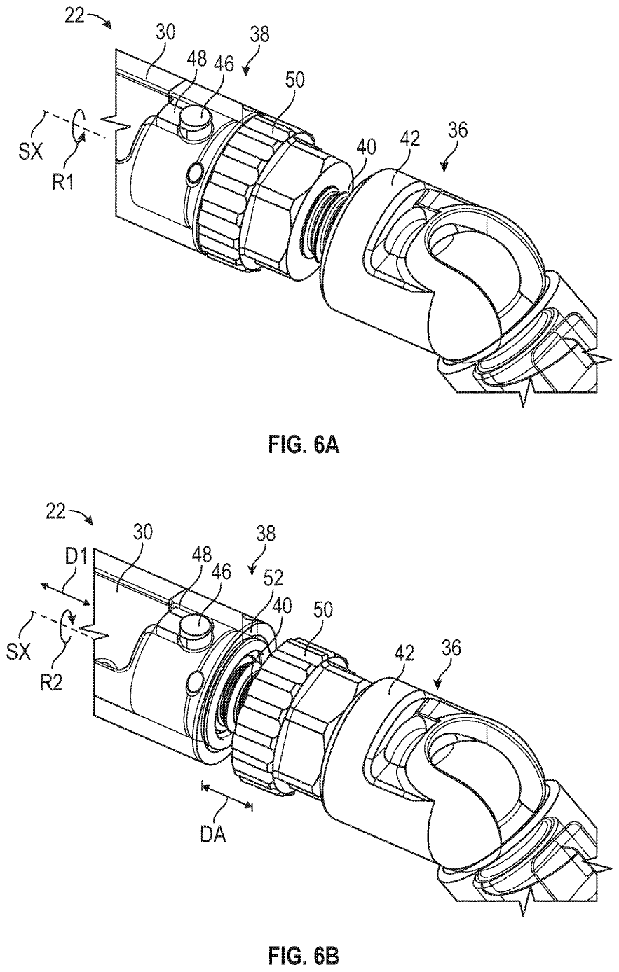

Referring to FIGS. 6A-6B, with continuing reference to FIGS. 3-5, the dynamization mechanism 38 may include a locked (e.g., fixed) mode and/or an unlocked (e.g., dynamization) mode. A position of the adjustment control 50 may be adjustable in the unlocked mode to vary an amount of permitted dynamization travel (e.g., collapse and/or expansion) of the strut 22. FIG. 6A may be associated with the locked mode. FIG. 6B may be associated with the unlocked mode. The strut body 30 may be moveable relative to the dynamization shaft 40 in the locked mode, but not in the unlocked mode.

When the patient places a load (e.g., weight) on the limb, the strut 22 may collapse when the dynamization feature 38 is unlocked. The amount of the collapse may be limited to the amount set by the adjustment control 50. The adjustment control 50 may be set to vary (e.g., increase or decrease) an amount of permitted dynamization travel (e.g., collapse and/or expansion) of the strut 22. The dynamization mechanism 38 may be adjusted over a (e.g., preselected) duration to gradually (e.g., incrementally) transfer a distribution of loads from the external fixation device 20 to the corrected bone.

The adjustment control 50 may be adapted to move relative to the dynamization shaft 40 to either lock or unlock dynamization of the strut 22 in the respective locked and unlocked modes. A position of the adjustment control 50 may be adjustable in the unlocked mode to set an axial rigidity of the strut 22. The adjustment control 50 may be rotatable about, or otherwise moveable along, the axis SX of the dynamization shaft 40 to vary a position of the adjustment control 50. The adjustment control 50 may be rotatable in a first (e.g., counterclockwise) direction R1 about the axis SX to move the dynamization mechanism 38 from the locked mode to the unlocked mode. The adjustment control 50 may be rotatable in a second (e.g., clockwise) direction R2 about the axis SX to move the dynamization feature 38 from the unlocked mode to the locked mode.

The adjustment control 50 may include a range of positions in the unlocked mode relative to the dynamization shaft 40. A body of the adjustment control 50 may be adapted to abut the end of the strut body 30 in a locked (e.g., minimum) position associated with the locked mode. In the unlocked mode, the strut 30 may be moveable relative to the dynamization shaft 40. The strut body 30 may be moveable in an (e.g., axial) direction D1 to vary a position of the strut body 30 relative to the strut axis SX. The body of the adjustment control 50 may be adapted to abut the joint body 42 of the joint assembly 36 in a fully unlocked (e.g., maximum) position associated with the unlocked mode. The strut body 30 may be adapted to translate (e.g., float or freely travel) along the dynamization shaft 40 in the unlocked mode such that the strut 22 may collapse or expand by a limited (e.g., controlled) amount. The strut body 30 may be moveable a distance DA relative to the strut axis SX. A permitted amount of dynamization travel (e.g., collapse and/or expansion) of the strut 22 may be associated with the distance DA, which may be set in response to manipulating the adjustment control 50. The distance DA may be established between the strut body 30 and the joint body 42 when the retention pin 46 may abut an end of the retention slot 48 (e.g., FIG. 6B). The strut body 30 may be configured to freely travel a maximum distance along the strut axis SX that may be substantially equal to the distance DA.

The spring member 52 may be adapted to oppose a collapse of the strut 22 in the unlocked mode. The spring member 52 may be adapted to oppose (e.g., axial) movement of the strut body 30 relative to the dynamization shaft 40 in the unlocked mode. The spring member 52 may be adapted to cause the strut 22 to expand in response to lessening a load on the strut 22.

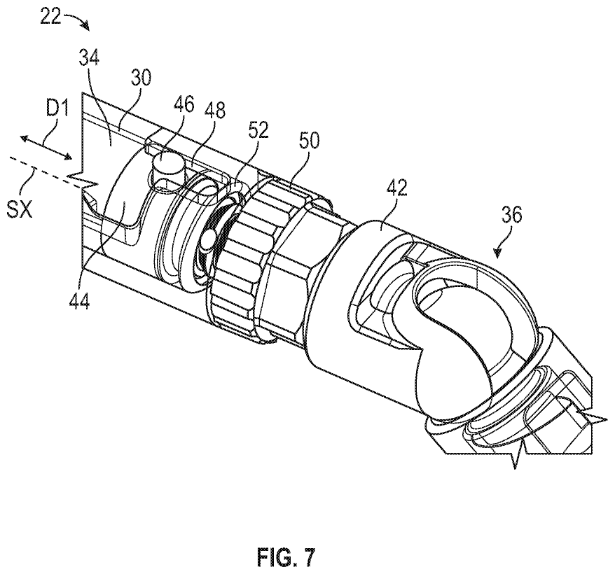

The retention pin 46 may be adapted to abut an end of the retention slot 48 to limit relative movement between the strut body 30 and the dynamization shaft 40. In the locked mode, the strut body 30 may be trapped between the retention pin 46 and the adjustment control 50 (e.g., FIG. 6A). In the unlocked mode, the strut body 30 may be moveable between a first boundary established by the retention pin 46 (e.g., FIG. 6B) and a second boundary established by the adjustment control 50 (e.g., FIG. 7). In the implementation of FIG. 7, the strut body 30 may be dimensioned to abut the adjustment control 50 to limit movement of the strut body 30 in the direction D1. The retention pin 46 may be spaced apart from the end of the retention slot 48 when the strut 22 is collapsed.

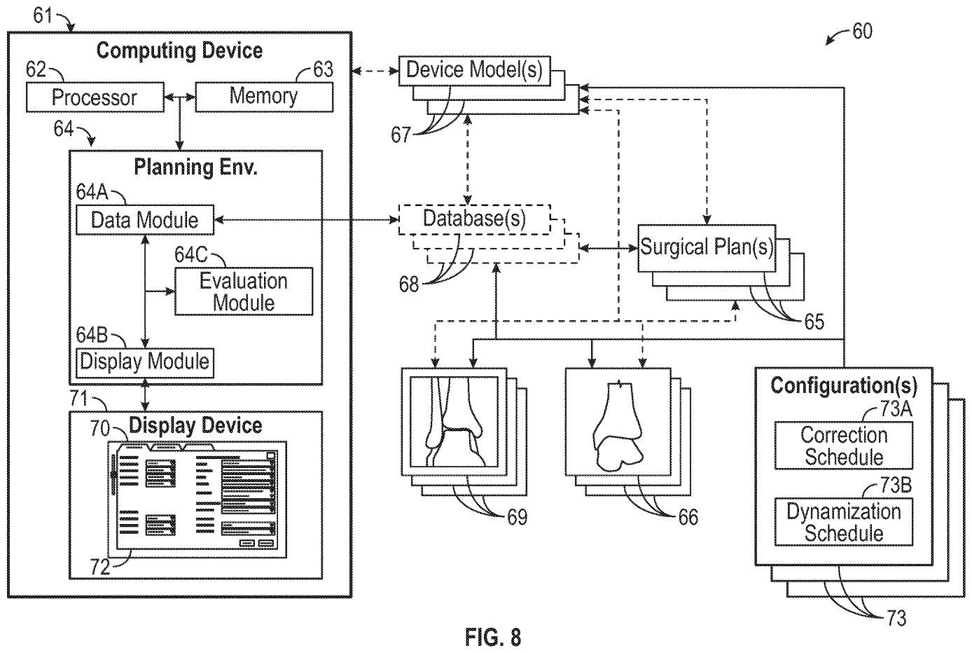

FIG. 8 discloses a planning system 60 for planning a surgical procedure according to an implementation. The system 60 may be utilized for planning orthopaedic and/or other surgical procedures, including pre-operatively, intra-operatively and/or post-operatively to create, edit, execute and/or review surgical plans. The system 60 may be utilized to plan and implement various orthopaedic and other surgical procedures, such as an arthroplasty to correct one or more bone deformities. The system 60 may be utilized in planning the preparation of one or more bones. The system 60 may be utilized in the placement and/or configuration of surgical devices, such as an external fixation system including the system 20 (e.g., FIG. 1). In implementations, the system 60 may be utilized to correct an angular deformity. One or more osteotomies (e.g., cuts or resections) may be established in the bone(s) to realign the bone and/or an associated limb. Although the planning systems and methods disclosed herein primarily refer to repair of a leg, it should be understood that the planning system 60 may be utilized in the repair of other anatomy and other surgical procedures including repair of other joints such as a shoulder, wrist, hand, hip, ankle or knee. Various bones may be treated, including a long bone such as a femur or tibia of a leg, and an ulna or radius of a forearm.

The system 60 may include one or more computing devices 61. The computing devices 61 may include one or more computer processors, memory, storage means, network devices, input and/or output devices and/or interfaces. The input devices may include keyboards, mice and touch screens. The output devices may include monitors, speakers and printers. The memory may include UVPROM, EEPROM, FLASH, RAM, ROM, DVD, CD, a hard drive, or other computer readable medium which may store data and/or other information relating to the planning and implementation techniques disclosed herein. The computing device 61 may be a desktop computer, laptop computer, smart phone, tablet, or any other computing device. The interface may facilitate communication with the other systems and/or components of a network.

The computing device(s) 61 may include one or more processors 62 operatively coupled to memory 63. The computing device 61 may be a standalone device. In implementations, the computing device 61 may be a client computer incorporated into a client-server architecture configured to execute computer software on a host computer, which may be accessible by the client computer using either a thin client application or a web browser executing on the client computer. The computing device 61 may be configured to interface with a cloud computing system. The computing device(s) 61 may be configured to execute one or more software programs, including various surgical tools.

The computing device(s) 61 may be operable to access and/or (e.g., locally or remotely) execute a planning environment 64. The planning environment 64 may be a standalone software package or may be incorporated into another surgical tool. The processor(s) 62 may be operable to collectively execute the planning environment 64. The planning environment 64 may be operable to create, edit, execute and/or review one or more surgical plans 65 and one or more associated anatomical (e.g., bone) models 66 and/or device (e.g., fixation) models 67, which may occur during pre-operative, intra-operative and/or post-operative phases of a surgery. The anatomical model 66 may be associated with one or more bones, joints and/or soft tissue of a patient. Each surgical plan 65 may be associated with the anatomy of a respective patient.

The planning environment 64 may include a data (e.g., interface) module 64A, a display module 64B and/or an evaluation (e.g., spatial) module 64C. Although three modules are disclosed, it should be understood that fewer or more than three modules may be utilized and/or one or more of the modules may be combined to provide the disclosed functionality.

The data module 64A may be configured to access, retrieve and/or store data and other information in one or more database(s) 68, which may include one or more images 69 of patient anatomy, anatomical model(s) 66, device model(s) 67 and/or surgical plan(s) 65. The data and other information may be stored in the database(s) 68 as one or more records or entries. In implementations, the data and other information may be stored in one or more files that may be accessible by referencing one or more objects or memory locations referenced by the records. Images 69, anatomical models 66, device models 67 and/or surgical plans 65 stored in the database(s) 68 may correspond to respective patient anatomies from prior, planned and/or hypothetical surgical cases, and may be arranged into one or more predefined categories such as sex, age, ethnicity, defect category, procedure type, surgeon, and/or facility or organization.

The memory 63 may be configured to access, load, edit and/or store instances of one or more images 69, anatomical models 66, device models 67 and/or surgical plans 65 in response to one or more commands from the data module 64A. The data module 64A may be configured to access the image(s) 69, bone model(s) 66, device model(s) 67 and/or surgical plan(s) 65 from memory, such as the memory 63 and/or a storage system. The data module 64A may be configured to cause the memory 63 to store a local instance of the image(s) 69, anatomical model(s) 66, device model(s) 67 and/or surgical plan(s) 65, which may be synchronized with the records in the database(s) 68.

The system 60 may include, or may be coupled to, one or more imaging devices. The imaging device may be configured to capture or acquire one or more images of patient anatomy residing within a scan field (e.g., window) of the imaging device. The imaging device may be configured to capture (e.g., acquire) two-dimensional (2D) and/or three-dimensional (3D) greyscale and/or color images. Various imaging devices may be utilized, such as an X-ray machine, computerized tomography (CT) machine or magnetic resonance imaging (MRI) machine that may obtain one or more images of a patient.

The planning environment 64 may be configured to interact with one or more imaging devices to capture or acquire images 69 of patient anatomy. The data module 64A may be configured to receive data and other information corresponding to at least one or more images 69 of patient anatomy from various sources including any of the imaging devices disclosed herein. The data module 64A may be configured to command the imaging device to capture or otherwise acquire the images 69 automatically and/or in response to user interaction.

Each image 69 and/or anatomical model 66 may include data and other information obtained from one or more medical devices or tools, such as the imaging device(s). The anatomical model 66 may include coordinate information relating to an anatomy of the patient obtained or derived from image(s) 69 obtained (e.g., captured) by the imaging device(s). Each device model 67 may include geometry and/or coordinate information associated with a (e.g., predefined) design or a design established (e.g., modified) by the planning environment 64.

Each surgical plan 65 may be associated with one or more images 69, anatomical models 66 and/or device models 67. The surgical plan 65 may include various parameters associated with the images 69, anatomical models 66 and/or device models 67. The parameters may include spatial information relating to relative positioning and coordinate information of the selected anatomical model(s) 66 and/or device model(s) 67.

The surgical plan 65 may include one or more revisions to the anatomical model 66 and information relating to placement of the device model 67 relative to the original and/or revised anatomical model 66. The revisions may include one or more cuts and/or holes. The surgical plan 65 may include coordinate information relating to the revised anatomical model 66 and a relative placement of the device model 67 in predefined data structure(s). Revisions to the anatomical model 66 and/or surgical plan 65 may be stored in the database 68 automatically and/or in response to user interaction with the system 60.

The planning environment 60 may be configured to provide a display or visualization of one or more images 69, virtual anatomical (e.g., bone) models 66 and/or virtual device models 67 in one or more graphical user interfaces (GUI) 70. The display module 64B may be configured to display data and other information relating to one or more surgical plans 65 in the user interface 70, including one or more of the images 69, anatomical models 66, and/or device models 67. The computing device 61 may incorporate, or may be coupled to, a display device 71. The user interface 70 may include one or more display windows 72. The display module 64B may be configured to cause the display device 71 to display information in the display window(s) 72 and/or another portion of the user interface 70. The planning environment 64 may incorporate and/or interface with one or more modeling packages, such as a computer aided design (CAD) package, to render the models 66, 67 as virtual 2D and/or 3D volumes or constructs, which may be displayed in the display window(s) 72 of the user interface 70. Each anatomical model 66 and/or device model 67 may correspond to 2D and/or 3D geometry and may be utilized to generate a wireframe, mesh and/or solid construct in the display window(s) 72. A surgeon or other user may interact with the user interface 70 via the planning environment 64 to view one or more images 69 of patient anatomy, anatomical models 66, and/or device models 67. The surgeon or other user may interact with the user interface 70 via the planning environment 64 to create, edit, execute and/or review one or more surgical plans 65.

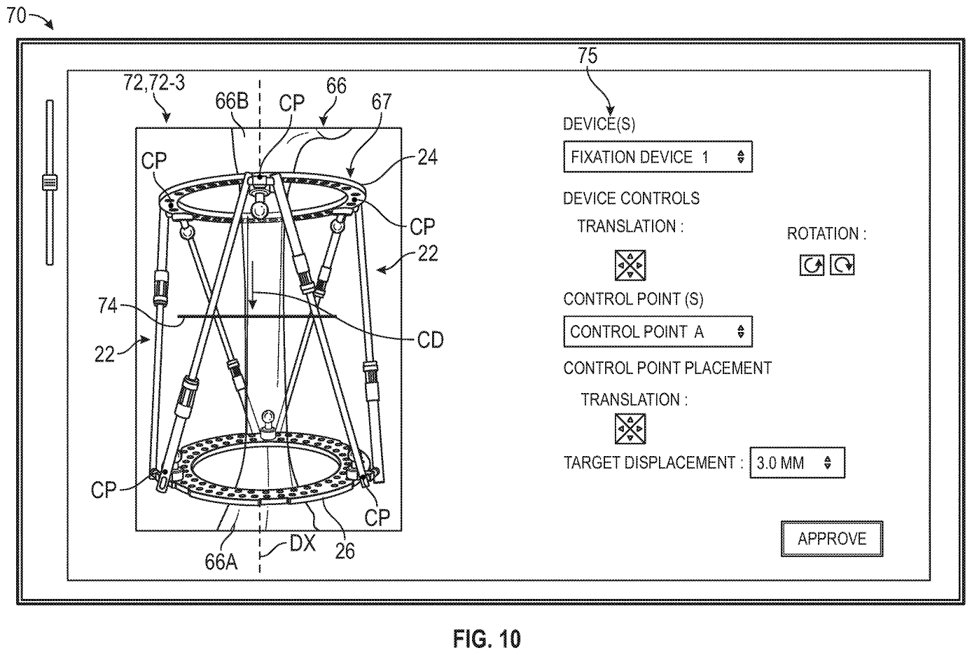

The planning system 60 may be configured to access, generate, review, edit and/or approve one or more configurations 73 associated with respective surgical device(s), including any of the devices disclosed herein such as the external fixation system 20. The device model 67 (e.g., FIG. 10) may be associated with an external fixation device. The device model 67 may be representative of a respective physical surgical device, such as a physical external fixation system 20 (e.g., FIGS. 1-2) and/or one or more struts 22 (e.g., FIG. 3). The configuration 73 may specify various information for configuring a physical instance of the fixation system 20, which may be based on a respective device model 67 and/or surgical plan 65 for treating a patient. In implementations, the configuration 73 may specify one or more settings (e.g., adjustments) associated with respective struts 22 of the external fixation system 20 (e.g., FIGS. 1-3). Each configuration 73 may include one or more files in a predetermined data structure or format.

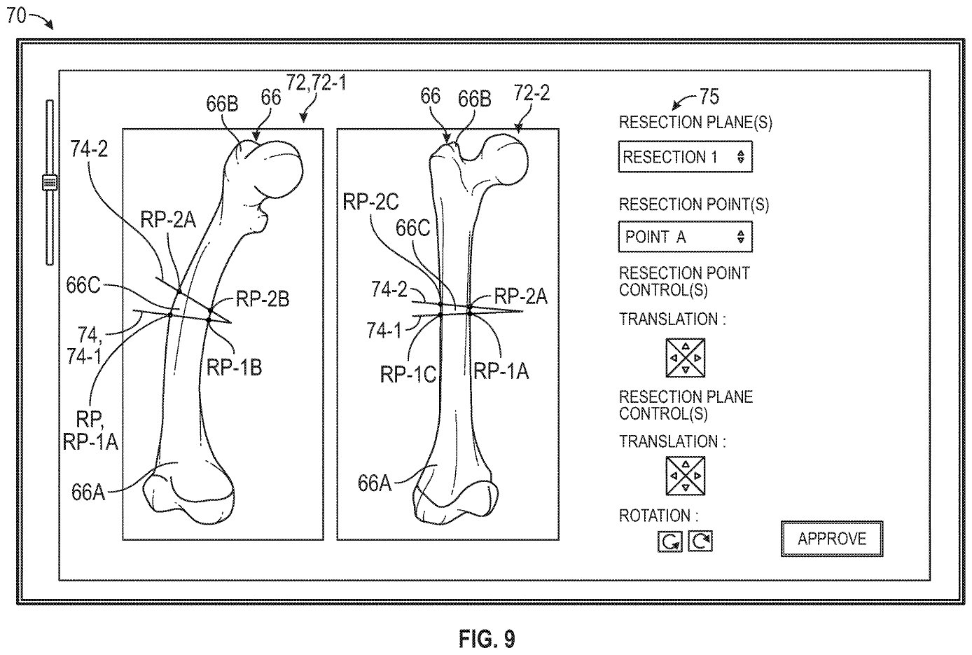

Referring to FIG. 9, with continuing reference to FIG. 8, the various techniques may be utilized to specify one or more resections (e.g., cuts or osteotomies) to correct a deformity of a bone, which may be associated with a respective anatomical (e.g., bone) model 66. Although this disclosure primary refers to a complete resection of the deformed bone, the disclosed techniques may be utilized in other surgical techniques including a partial resection such as an open-wedge or closed-wedge osteotomy. The partially resected bone may be pivoted about a pivot point to perform the deformity correction. The resection may have various geometries, such as a planar or curved cut.

The user interface 70 may include a first display window 72-1 and/or a second display window 72-2. The display windows 72-1, 72-2 may be operable to display the selected bone model 66 at different orientations relative to each other. The orientations may be associated with one or more kinematic planes of the patient anatomy. In implementations, the display windows 72-1, 72-2 may be operable to display views of the bone model 66 at 90 degrees relative to each other.

The planning environment 64 may be operable to establish one or more osteotomy (e.g., resection) planes 74 along the (e.g. three-dimensional) bone model 66, which may be associated with an osteotomy (e.g., resection) of a bone. The bone model 66 may be associated with a (e.g., deformed) bone of a patient. The resection plane 74 may be associated with a resection that may divide the bone into a first portion fixed (e.g., attached to) the first support 24 and/or a second portion fixed (e.g., attached to) the second support 26. In the implementation of FIG. 9, the resection planes 74 may include a first resection plane 74-1 and/or a second resection plane 74-2. The resection planes 74-1, 74-2 may be non-parallel to each other. The resection planes 74 may establish two or more (e.g., discrete) portions of the bone model 66. In the implementation of FIG. 9, the resection planes 74 may divide a volume of the bone model 66 into first, second and/or third bone portions 66A-66C. The deformity correction may include the removal of the third portion 66C. First and second bone portions (e.g., segments) 66A, 66B of the bone model 66 may be on opposite sides of the resection plane 74. The bone portions 66A, 66B may be rearranged relative to each other to correct a deformity. The deformity correction may include placing the bone portions 66A, 66B in abutment along the resection surfaces, which may be according to a specified orientation (e.g., FIG. 10). The bone portions B1, B2 of the bone B may be associated with the bone portions 66A, 66B, and the resection R may be associated with the resection plane 74 (e.g., FIG. 2).

The user interface 70 may be adapted to establish one or more resection planes 74 relative to the bone model 66. Various techniques may be utilized to establish a position and/or orientation of the resection plane(s) 74. In implementations, the surgeon or clinical user may interact with menu(s) 75 of the user interface 70. Each resection plane 74 may be associated with one or more resection points RP. The resection points RP may overlay the respective display window 72. In implementations, the resection points RP may include a set of three resection points. The planning environment 64 may be operable to establish one or more resection planes 74 based on a respective set of the resection points RP, which may be along the bone model 66. The resection points RP may be set in response to user interaction with the user interface 70. In implementations, the resection points RP associated with the first resection plane 74-1 may include resection points RP-1A to RP-1C. The second resection plane 74-2 may be associated with resection points RP-2A to RP-2C. The surgeon or clinical user may interact with the menu 75 and/or directly with the display window(s) 72-1, 72-2 to set a position of the respective resection points RP relative to the bone model 66. The display windows 72-1, 72-2 may be dynamically linked such that movement of a resection point RP in one of the display windows 72-1/72-2 may cause a change in the position of the same resection point RP in the other display window 72-1/72-2. In implementations, the surgeon or clinical user may select two resection points RP near the resection plane 74 in an anterior-posterior view and may select one resection point RP at a level of the resection plane 74 in a medial-lateral view, or vice versa.

Other techniques may be utilized to establish the resection planes 74. In implementations, the surgeon or clinical user may interact with the display windows 72-1, 72-2, with the menu 75 and/or other portion of the user interface 70 to specify a pair of lines associated with the respective views of the bone model 66. The evaluation module 64C may establish the resection plane 74 in three-dimensional space based on placement of the lines.

Referring to FIG. 10, with continuing reference to FIGS. 8 and 9, the planning environment 64 may be operable to display the device model 67 relative to uncorrected and/or corrected instances of the bone model 66 in one or more display windows 72 of the user interface 70. The planning environment 64 may be operable to place the device model 67 about a periphery of the bone model 66. The planning environment 64 may be operable to display the device model 67 relative to the bone model 66. The user interface 70 may include a third display window 72-3. The planning environment 64 may be operable to display the resection plane 74 relative to the bone model 66. The evaluation model 64C may be operable to place the device model 67 relative to the bone model 66. The device model 67 may be associated with an external fixation device, such as the fixation system 20 of FIGS. 1-2. The device model 67 may include any of the structure of the fixation system 20. The device model 67 may include one or more struts 22, which may interconnect first and second supports 24, 26. The struts 22 may be distributed about a device (e.g., longitudinal) axis DX and may interconnect the supports 24, 26. The positions of the supports 24, 26 may be set (e.g., fixed) relative to the respective first and second portions 66A, 66B of the bone model 66.

The planning environment 64 may include one or more features for setting the placement (e.g., position and/or orientation) of the device model 67. The surgeon or clinical user may interact with a menu 75, the display window 72-3 and/or another portion of the user interface 70 to set the position and/or orientation of the device model 67 and/or one or more components of the device model 67, including moving the device model 67 in two-dimensional and/or three-dimensional space.

The surgeon or clinical user may interact with the user interface 70 to set one or more control points CP associated with the device model 67. The control points CP may be established in two-dimensional and/or three-dimensional space. Each strut 22 may be associated with a respective set of the control points CP. Control points CP may be established for the interfaces (e.g., joints) between the struts 22 and the supports 24, 26. The joint assemblies 36 may be associated with respective control points CP. In implementations, an adjacent pair of struts 22 may be associated with a common control point CP along the respective ring 24, 26. The user may interact with the display window 72-3, the menu 75 and/or another portion of the user interface 70 to set a position of the control point CP and the associated structure of the device model 67. The control points CP may be dynamically linked to each other such that the adjustment of the position of one control point CP may cause the adjustment of one or more other control points CP associated with the respective structure including the supports 24, 26 based on a geometry of the device model 67. A length of the strut 22 may be varied in response to changing a position of the control point(s) CP and/or vice versa.

The positions of the control points CP may be established for a first (e.g., initial) position and a second (e.g., final) position of the device model 67. The initial position may be associated with an arrangement of the external fixation device 20 prior to the deformity correction (e.g., start of prescription). The final position may be associated with an arrangement of the external fixation device 20 at completion of the deformity correction (e.g., end of prescription). The length of the struts 22 to achieve the deformity correction may be determined based on the positions of the control points CP with respect to the initial and final positions.

In the implementation of FIG. 8, the planning environment 64 may be operable to establish one or more configurations 73 associated with the respective device model 67. The configuration 73 may include one or more settings (e.g., adjustments).

The configuration 73 may include a correction schedule 73A and/or a dynamization schedule 73B. The planning environment 64 may be operable to establish the correction schedule 73A and/or dynamization schedule 73B. The correction schedule 73A may be associated with a relative position between the resected portions of the bone before and after the deformity correction (e.g., start and end of prescription). The correction schedule 73A may be associated with one or more sets of adjustments that may vary the lengths of the struts 22 to establish a correction of the respective bone, which may occur over a (e.g., preselected) duration. The evaluation module 64C may be operable to determine the adjustments based on the relative positions of the supports 24, 26 prior to and upon completion of the deformity correction. The correction may cause the resection surfaces associated with the resection planes 74-1, 74-2 to abut each other in a (e.g., preselected) orientation. The adjustments may establish a compressive force along the resection surface.

The dynamization schedule 73B may be implemented to establish a controlled amount of dynamization travel (e.g., collapse and/or expansion) of the strut 22 and a controlled collapse of the associated external fixation device 20 during dynamization, which may improve healing of the corrected bone. Once the correction schedule 73A and associated correction of the deformity may be completed, the dynamization schedule 73B may be executed to transfer a distribution of loads (e.g., weight) from the external fixation device 20 to the corrected bone during dynamization. The dynamization schedule 73B may include one or more sets of adjustments that may vary (e.g., increase or decrease) an amount of permitted dynamization travel (e.g., collapse and/or expansion) of the struts 22, which may occur over a (e.g., preselected) duration. The surgeon or clinical user may interact with the dynamization feature 38 to adjust the struts 22 according to the dynamization schedule 73B.

The struts 22 may be adjustable to (e.g., collectively) establish a direction of collapse CD of the device model 67 and/or associated external fixation device 20 (e.g., FIG. 2). The dynamization schedule 73B may include adjustments that may (e.g., incrementally) vary an amount of permitted dynamization travel (e.g., collapse and/or expansion) of the struts 22, which may occur over a (e.g., preselected or prescribed) duration. One or more of the adjustments associated with the dynamization schedule 73B may cause a change (e.g., reduction or increase) in the axial rigidity of the respective struts 22. The dynamization schedule 73B may include a respective set (e.g., combination) of adjustments for the prescribed duration, which may occur over day(s) and/or week(s). The adjustments may introduce a controlled allowance for dynamization travel (e.g., axial displacement) in one or more of the struts 22. The adjustments may include reductions in the axial rigidity of at least one or more, or all, of the struts 22. The set of adjustments may cause an increase in the axial rigidity of one or more, or all, of the struts 22, while other adjustments may cause a reduction in the axial rigidity of one or more of the remaining struts 22. The adjustments may include different amounts associated with the respective struts 22. The different amounts may include adjustments over a single day and/or different amounts over a set of days during the dynamization schedule 73B. The adjustments may vary the amount of permitted dynamization travel of the struts 22 such that the direction of collapse CD may be substantially parallel to the displacement vector 76 in response to the adjustments, which may be performed during execution of the dynamization schedule 73B. The device axis DX may be substantially parallel to the direction of collapse CD.

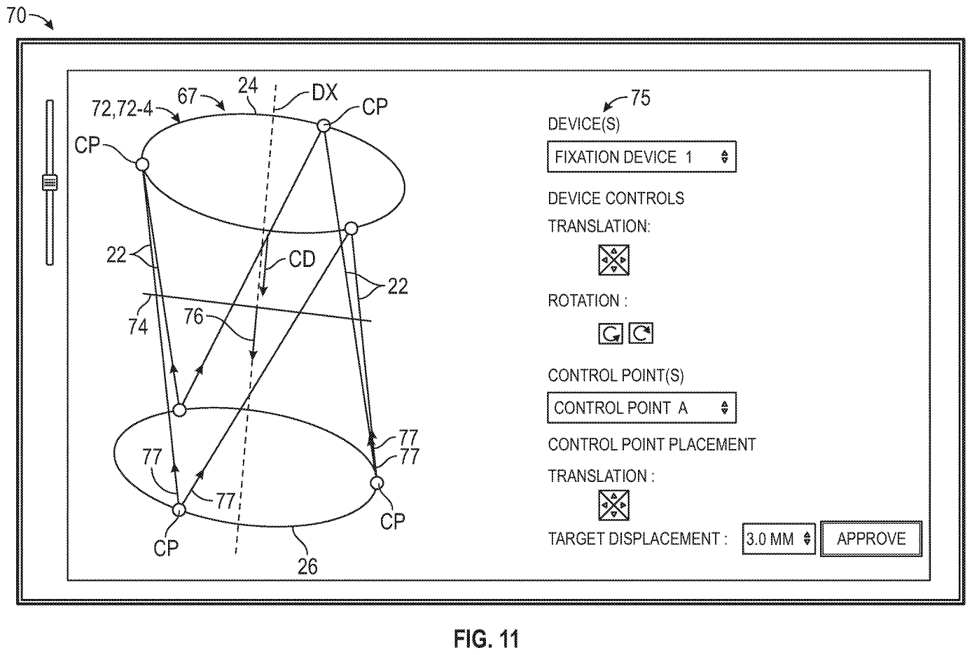

Referring to FIG. 11, with continuing reference to FIGS. 1-3 and 8-10, the planning environment 64 may be operable to establish one or more characteristics of the device model 67, which may be associated with dynamization of a bone associated with the bone model 66. The interface 70 may include a fourth display window 72-4. The display window 72-4 may be operable to display the device model 67 and/or a representation of the device model 67. In the implementation of FIG. 11, the representation may include a wire frame of the device model 67. The surgeon or clinical user may interact with a menu 75, the display window 72-4 and/or another portion of the user interface 70 to set the position of one or more control points CP associated with the device model 67. The display window 72-4 may be operable to display the resection plane(s) 74, including relative to the device model 67.

The surgeon or clinical user may interact with the user interface 70 to specify one or more settings associated with the device model 67, including a target displacement (e.g., dynamization). The target displacement may be associated with a controlled (e.g., interfragmentary) movement between adjacent bone portions of a resected bone. The movement may establish compression between the bone portions, which may promote bone fusion. The surgical plan 65 may include a target displacement value associated with correction of a bone deformity. The surgeon or clinical user may interact with the user interface 70 to specify the target displacement value. The target displacement value may be associated with a compression of the bone at an end of correction (e.g., prescription). The compression may be adjacent to a resection, which may be associated with the resection plane 74. The surgical plan 65 may include a dynamization schedule for the corrected bone based on the target displacement value.

The bone model 67 may be associated with a (e.g., target) displacement vector 76. The evaluation module 64C may be operable to determine the displacement vector 76, which may be associated with compression of the bone at the end of correction (e.g., prescription). The displacement vector 76 may be associated with compression of the bone adjacent to the resection, which may be associated with the resection plane 74, during consolidation of the bone subsequent to correction of the deformity. In implementations, the displacement vector 76 may be defined according to the target displacement value, which may be specified in the user interface 70. In implementations, the displacement vector 76 may be established such that the displacement vector 76 may be substantially normal to the resection plane 74.

Various techniques may be utilized to determine the dynamization schedule 73B, which may be implemented during consolidation of a corrected bone. The dynamization schedule 73B may be established based on the target displacement value. The dynamization schedule 73B may be established such that a device (e.g., longitudinal) axis DX of the fixation model 67 may be (e.g., remain) substantially parallel to the displacement vector 76 and/or an osteotomy (e.g., resection) plane normal during a duration of the dynamization schedule 73B.

The struts 22 may be associated with respective strut (e.g., unit) vectors. The device model 67 may be associated with a set of strut vectors 77, which may be associated with the struts 22. The evaluation model 64C may be operable to determine the strut vector(s) 77. The strut vectors 77 may be established with respect to the displacement vector 76.

The strut vectors 77 may be based on a geometry and/or arrangement of the supports 24, 26 and struts 22. The strut vector 77 may have an origin at a connection between the strut 22 and support 24/26. The strut vectors 77 may be associated with the orientation of the struts 22 relative to the support 24/26 at the completion of the deformity correction (e.g., end of prescription). The evaluation module 64C may be operable to update the strut vectors 77 based on a change in the length(s) of the struts 22, which may occur due to a change in the prescription.

The orientation of the device model 67 may be established such that the device axis DX may be substantially parallel to a direction of the displacement vector 76. The evaluation module 64C may be operable to determine one or more sets (e.g., combinations) of adjustments which may vary the amounts of permitted dynamization travel (e.g., collapse and/or expansion) of the struts 22 based on the respective strut unit vectors 77 and/or the target displacement value such that the device axis DX may remain substantially parallel to the displacement vector 76 in response to the respective combination(s) of adjustments. The dynamization schedule 73B may include one or more sets (e.g., combinations) of adjustments that may vary the axial rigidity of the struts 22 based on the respective strut vectors 77 and/or the target displacement such that the device axis DX of the fixation model 67 may be (e.g., remain) substantially parallel to the displacement vector 76 during a preselected duration. Some (or all) struts 22 may be adjusted during a single adjustment period while others may not. The struts 22 may be adjustable to vary the axial rigidity. The combination(s) of adjustments may include different amounts that may vary the permitted dynamization travel of the struts 22.

Various techniques may be utilized to determine adjustments to the dynamization mechanisms 38 for achieving the prescribed dynamization and a controlled collapse of the external fixation device 20. The evaluation module 64C may be operable to determine combination(s) of adjustments to the dynamization feature(s) 38 of the struts 22 to achieve the prescribed dynamization based on regression, such as a least-squares regression computation. The regression may be based on the displacement vector 76 and/or the strut unit vectors 77. The least-squares regression computation may be associated with the displacement vector 76 and/or strut vectors 77. The evaluation module 64C and/or another portion of the planning environment 64 may be operable to determine dynamization using any of the techniques disclosed herein, including regression. Another technique for determining the adjustments may include a geometric projection, which may include calculating a dot product of the strut vectors 77 and the prescribed osteotomy normal vector (e.g., unit vector times magnitude). The regression technique may include checking the determinant and may include checking for unsolvable frame geometries (e.g., all struts parallel), but the geometric projection technique may not.

The regression computation may include determining a target (e.g. prescribed) displacement value. The target displacement value may be specified (e.g., prescribed) by the surgeon or clinical user. The target displacement value may be associated with compression caused by reducing a relative position between the supports 24, 26 when fixed to the bone. The amount of compression may be established at an end of correction (e.g., prescription) of the deformity based on the surgical plan 65 and/or associated correction schedule 73A. The bone may compress a lesser amount than the target displacement value. The compression may be established between the resection surfaces of adjacent portions of the corrected bone. The target displacement value may be specified in millimeters (mm). The target displacement value may be associated with a target compression in a normal vector to the resection (e.g., osteotomy) plane 74. The following values should not be understood as limiting. An implementation of the target displacement value is as follows:

| | |
|---|---|
| Target Displacement (mm) | 3.000 |

The regression computation may include determining (e.g., establishing) a strut matrix (A) associated with the strut vectors 77. The strut vectors 77 may be associated with a configuration of the fixation system at the end of correction. The following implementation of the regression computation may be based on a hexapod, which may be associated with a device model 67 having a total of six struts 22. The following values are associated with a hexapod implementation and should not be understood as limiting. The strut matrix (A) may be a six-by-three matrix as follows:

| | Description | X | Y | Z |
|---|---|---|---|---|
| Matrix (A) | Strut 1 | −0.065 | 0.242 | 0.967 |
| | Strut 2 | −0.242 | 0.065 | 0.967 |
| | Strut 3 | −0.177 | −0.177 | 0.967 |
| | Strut 4 | 0.065 | −0.242 | 0.967 |
| | Strut 5 | 0.242 | −0.065 | 0.967 |
| | Strut 6 | 0.177 | 0.177 | 0.967 |

The z-direction may be parallel to the device axis DX of the device model 67. The x-axis and y-axis may be perpendicular to the z-direction. The x-axis and y-axis may extend along a plane of the support 24/26.

The regression computation may include determining (e.g., establishing) an osteotomy (e.g., resection) normal unit vector. The osteotomy normal unit vector may be normal to the resection plane 74. An implementation of the osteotomy normal unit vector may be as follows:

| Description | X | Y | Z |
|---|---|---|---|
| Osteotomy Normal | 0.000 | 0.000 | 1.000 |

An osteotomy normal unit vector having a value of 1.0 in the z-direction may be associated with a resection plane 74 having components in the x- and y-directions but not in the z-direction. The disclosed techniques may be utilized for resection planes 74 having components in the x-, y- and/or z-directions.

The regression computation may include determining (e.g., establishing) a target displacement vector 76. The target displacement vector 76 may be associated with a magnitude of the target displacement and a (e.g., desired) direction of motion of the support 24/26 and/or compression. The target displacement vector 76 may be expressed as a positive or negative value and indicates the amount of compression. The target displacement vector 76 may be expressed in a three-by-one matrix (e.g., x, y and z directions) as follows:

| | | |
|---|---|---|
| Target displacement | X | 0.000 |
| vector (mm) | Y | 0.000 |
| | Z | 3.000 |

The regression computation may include determining (e.g., establishing) a Gram matrix (A\hat{ }T*A) based on the strut matrix A. The Gram matrix may be a symmetric matrix of inner products between the strut unit vectors 77. The Gram matrix may characterize how the struts 22 all interact as a system, including the relative orientation of the struts 22 in three-dimensional space. The Gram matrix may be a three-by-three matrix as follows:

| | | | |
|---|---|---|---|
| Matrix (A\hat{ }T*A) | 0.188 | 0.000 | 0.000 |
| | 0.000 | 0.188 | 0.000 |
| | 0.000 | 0.000 | 5.611 |

The Gram matrix may be used to apply motion to the struts 22 to achieve motion of the external fixation device 20 that may be parallel to the target displacement vector 76.

The regression computation may include determining (e.g., computing) a determinant. The determinant may be a Gram matrix determinant (e.g., Gramian). The Gram determinant may be expressed as det(A\hat{ }T*A). The Gram determinant may be defined as the volume scaling factor of the transformation defined by the strut unit vectors 77. If the determinant is greater than zero, the vectors 77 may be linearly independent and may be solved. If the determinant equals zero, then the vectors 77 may be coplanar or collinear, and collapse and/or expansion of the strut 22 may not be directed purely along the displacement vector 76. In implementations, a value of the determinant may be as follows:

| | |
|---|---|
| Determinant, | 0.199 |
| det(A\hat{ }T*A) | |

The determinant may be used in combination with the Gram matrix to determine (e.g., establish) a distribution of the movement of the struts 22 such that the movement of the external fixation device 20 may be in a direction that may be substantially parallel to the target displacement vector 76 as a system. The determinant may be associated with a distribution of movement of the struts 22 to collectively establish the direction of collapse CD of the device model 67 and/or associated external fixation device 20.

The regression computation may include determining (e.g., establishing) an inversion of the Gram matrix ((A\hat{ }T*A)\hat{ }−1). The inversion of the Gram matrix may be used to determine an (e.g., optimal) distribution of the (e.g., desired) movement of the fixation system 20.

The inversion of the Gram matrix may be based on the determinant. The inversion of the Gram matrix ((A\hat{ }T*A)\hat{ }−1) may be a three-by-three matrix as follows:

| | | | |
|---|---|---|---|
| Matrix | 5.312 | 0.007 | 0.000 |
| ((A\hat{ }T*A)\hat{ } − 1) | 0.007 | 5.312 | 0.000 |
| | 0.000 | 0.000 | 0.178 |

The regression computation may include determining (e.g., establishing) a pseudo-motion (e.g., intermediate) vector based on a product of the inversion of the Gram matrix ((A\hat{ }T*A)\hat{ }−1) and the target displacement vector 76. The pseudo-motion vector may establish a set of best-fit translation coefficients. The pseudo-motion vector may be expressed in a three-by-one matrix (e.g., x, y and z directions) as follows:

| | | |
|---|---|---|
| Pseudo-motion | X | 0.000 |
| vector (mm) | Y | 0.000 |
| | Z | 0.535 |

The best-fit translation coefficients may be applied to the respective strut unit vectors 77 to achieve a controlled collapse of the fixation system 20 that may in a direction substantially (e.g., purely) parallel to the target displacement vector 76.

The amount of dynamization may be proportional to the target displacement value and associated amount of compression. In implementations, an increase in the target displacement value may cause an increase in the amount of dynamization. A rigidity of the external fixation device 20 may reduce over a duration of the deformity correction. The amount of dynamization may account for the reduced rigidity.

The regression computation may include determining (e.g., establishing) an amount of dynamization of the respective struts 22 based on a product of the pseudo-motion vector and the respective strut vectors 77. The dynamization (e.g., adjustment) values for each strut 22 may be determined by projecting the pseudo-motion vector onto the strut unit vectors 77. The dynamization amounts may be as follows:

| | Dynamization |
|---|---|
| Description | Amount (mm) |
| Strut 1 | 0.517 |
| Strut 2 | 0.517 |
| Strut 3 | 0.517 |
| Strut 4 | 0.517 |
| Strut 5 | 0.517 |
| Strut 6 | 0.517 |

The dynamization (e.g., adjustment) amounts may be positive or negative. A positive value may denote collapse/shortening of the strut, and a negative value may denote extension/lengthening, or vice versa. The dynamization amounts may be the same when the supports 24, 26 may be parallel to each other at the end of correction. The dynamization amounts may differ when the supports 24, 26 may be non-parallel to each other at the end of correction. In implementations in which the target displacement vector 76 does not extend purely in the z-direction, then some struts 22 may be configured to collapse while other struts 22 may be configured to expand such that the device axis DX of the fixation device 20 (e.g., FIG. 1) may remain substantially parallel to the displacement vector 76 during consolidation of the corrected bone. The dynamization amounts may be for a single adjustment period (e.g., in the preceding table). The dynamization amounts may be used to determine total dynamization displacements of the struts 22 for the total duration of the dynamization schedule 73B, which may be allocated to a set of adjustment periods.

The regression computation may include determining (e.g., establishing) an amount of total dynamization displacement for each strut 22. The determined dynamization amount may be used as a scale factor for achieving the total dynamization displacement for the strut 22. The amount of total dynamization displacement for the respective strut 22 may be calculated by dividing a magnitude of the target displacement or associated target displacement vector 76 (e.g., 3 mm) by a magnitude of the pseudo-motion vector (e.g., 0.535 mm). The calculated value (e.g., 5.607) may be multiplied by the determined dynamization amount for the strut 22 (e.g., 0.517 mm) to compute the total dynamization displacement (e.g., 2.899 mm). An implementation of the amounts of total dynamization displacement for the struts 22 may be as follows:

| | Total Dynamization |
|---|---|
| Description | Displacement (mm) |
| Strut 1 | 2.899 |
| Strut 2 | 2.899 |
| Strut 3 | 2.899 |
| Strut 4 | 2.899 |
| Strut 5 | 2.899 |
| Strut 6 | 2.899 |

In implementations of a symmetric system (e.g., frame), the amounts of total dynamization displacement may be the same for all of the struts 22. In implementations of a non-symmetric system, the amounts of total dynamization displacement for the struts 22 may differ from each other.

The determined amounts of total dynamization displacement for the struts 22 may be allocated to the dynamization schedule 73B. The total dynamization displacement amounts may be implemented by one or more sets (e.g., combinations) of adjustments of the struts 22. The adjustment amounts may be the same or may differ from each other, including within a combination of adjustments to the struts 22. The evaluation module 64C may be operable to allocate the determined amounts of total dynamization displacement of the struts 22 to the dynamization schedule 73B. The adjustment amounts per adjustment period to achieve the total dynamization displacement may be allocated based on various techniques, such as a percentage of the total dynamization displacement. The adjustment amounts may be graduated. In implementations, the adjustments may be approximately four turns per day of the adjustment assembly 50 at 0.25 mm per turn. In use, the surgeon may adjust the dynamization features 38 of the struts 22 to set the amount of permitted dynamization travel according to the determined adjustment amounts such that a direction of collapse of the fixation device 20 may be substantially (e.g., purely) parallel to the target displacement vector 76, which may be normal to the resection plane. The dynamization schedule 73B may include one or more tables or lists specifying the prescribed adjustments.

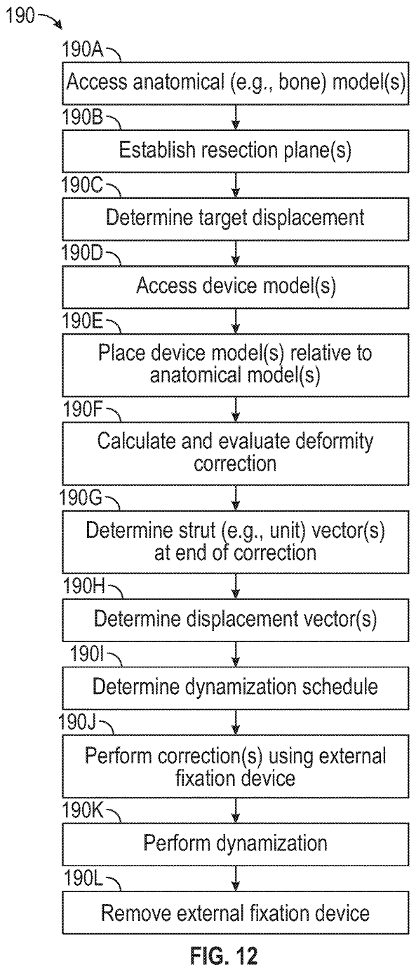

FIG. 12 discloses a method of planning and/or performing an orthopaedic procedure in a flowchart 190. The method 190 may be utilized pre-operatively, intra-operatively and/or post-operatively to create, edit, execute and/or review a surgical plan, which may be established for a patient. The method 190 may be utilized to perform an arthroplasty for restoring functionality to one or more bones and/or associated joints, including any of the bones and joints disclosed herein. The method 190 may be utilized in the correction of one or more deformed bones. The method 190 may be utilized with any of the planning systems disclosed herein. Fewer or additional steps than are recited below could be performed within the scope of this disclosure, and the recited order of steps is not intended to limit this disclosure. Reference is made to the planning system 60 and external fixation system 20.

Referring to FIG. 8, with continuing reference to FIG. 12, one or more anatomical (e.g., bone) models 66 may be accessed at block 190A. The anatomical model 66 may be associated with associated with one or more bones of a patient.

Referring to FIG. 9, with continuing reference to FIG. 12, one or more resection planes 74 may be established along the anatomical model 66 at block 190B. The resection plane(s) 74 may be established using any of the techniques disclosed herein.

Referring to FIG. 10, with continuing reference to FIGS. 1-3 and 12, a target displacement may be determined (e.g., established or prescribed) at block 190C. The target displacement may be associated with compression of the bone during a deformity correction. The compression may be along a resection associated with the resection plane 74. Block 190C may include determining a target displacement value (e.g., magnitude). Block 190C may include determining a displacement vector 76, which may be associated with compression of the bone during consolidation. The displacement vector 76 may be substantially normal to the resection plane 74.

At block 190D, one or more device models 67 may be accessed. The device model 67 may include any of the device models disclosed herein. The device model 67 may be associated with an external fixation device, including any of the fixation systems disclosed herein such as the fixation system 20, and may include a plurality of struts 22 interconnecting first and second supports 24, 26. The supports 24, 26 may be securable to bone.

At block 190E, the device model 67 may be placed relative to the anatomical model 66. The device model 67 may be placed and/or configured using any of the techniques disclosed herein.

At block 190F, a planned (e.g., deformity) correction associated with the device model 67 and/or associated fixation device 20 may be calculated and/or evaluated. The evaluation module 64C and/or another portion of the planning environment 64 may be operable to calculate and/or evaluate the correction. The correction may include a position and/or orientation of the adjacent bone portions 66A, 66B relative to each other and/or the resection plane 74 (e.g., FIGS. 9-10). The correction may be associated with abutment of the bone portions B1, B2 along the resection plane R based on the planned correction (e.g., FIG. 2). The correction may be established based on placement of the device model 67 relative to the portions 66A, 66B and a configuration of the device model 67, which may be specified by the configuration 73 (FIG. 8).

At block 190G, a set of strut (e.g., unit) vectors 77 may be determined (e.g., established). The strut vectors 77 may be determined for an arrangement of the device models 67 and/or associated fixation device 20 at the end of correction, which may be determined at block 190F. The strut vectors 77 may be associated with the respective struts 22. At block 190G, one or more target displacement vectors 76 may be determined (e.g., established). The vectors 76, 77 may be determined using any of the techniques disclosed herein.

At block 1901, a dynamization schedule 73B may be established. The dynamization schedule 73B may be implemented to permit (e.g., cause) a controlled collapse and/or expansion of the struts 22 and/or a controlled collapse of the external fixation device 20 over a (e.g., preselected) duration. The struts 22 may (e.g., collectively) establish a direction of collapse CD of the external fixation device 20, which may occur in response to the adjustment(s). The planning environment 64 may establish the dynamization schedule 73B, which may be based on one or more parameters specified by the surgeon or clinical user including a target displacement. The dynamization schedule 73B may include one or more adjustments that may vary an axial rigidity and/or an amount of permitted dynamization travel (e.g., collapse and/or expansion) of the respective struts 22. The adjustments may be based on the respective strut unit vectors 77 and/or the displacement vector 76 such that the direction of collapse CD and/or the device axis DX of the fixation device 20 may be (e.g., remain) substantially parallel to the displacement vector 67, which may include a (e.g., preselected) duration and/or during the consolidation of the bone.

Establishing the dynamization schedule 73B may include determining the adjustments based on (e.g., least-squares) regression. The regression may be based on the strut unit vectors 77 and/or the displacement vector 76. A strut matrix may be established. The strut matrix may be associated with the strut unit vectors 77. A Gram matrix and/or (e.g., Gram matrix) determinant may be established. The Gram matrix may be based on the strut matrix. The determinant may be associated with a distribution of movement of the struts 22 to collectively establish the direction of collapse CD. The Gram matrix may be inverted. The inverted Gram matrix may be based on the determinant. A pseudo-motion vector may be determined (e.g., solved) based on the inverted Gram matrix and/or the displacement vector 76. Amount(s) of dynamization (e.g., adjustments) of the struts 22 may be determined (e.g., computed) based on a product of the pseudo-motion vector and strut unit vectors 77. An amount of total dynamization displacement for each respective strut 22 may be determined. The amount of total dynamization displacement may be calculated based on the target displacement, pseudo-motion vector and/or determined dynamization amount.

The dynamization schedule 73B may have a (e.g., preselected) duration and may include a single adjustment period or a set of adjustment periods. The amount of total dynamization displacement may be allocated over the duration of the dynamization schedule 73B. The adjustments associated with the respective struts 22 may be established based on the allocations.

The surgeon may implement the surgical plan 65 to treat the patient. Implementing the surgical plan 65 may include performing the correction(s) to the bone at block 190J. The correction(s) may be performed using the external fixation device 20. The correction(s) may be performed according to the correction schedule 73A. Performing the correction(s) may include resecting the bone, placing the external fixation device 20 relative to the bone, securing (e.g., fixing) the external fixation device 20 to the resected bone, moving adjacent portions of the bone relative to each other, and/or causing the bone portions to compress against each other based on the target displacement, which may be specified in the surgical plan 65.

Implementing the surgical plan 65 may include performing dynamization at block 190K. The dynamization may be performed according to the dynamization schedule 73B. The dynamization schedule 73B may be followed to establish a controlled collapse of the external fixation device 20, which may transfer loads from the fixation device 20 to the bone during consolidation. The struts 22 may be adjusted during consolidation of the bone, which may be according to the dynamization schedule 73B. Performing the adjustments may include moving a dynamization adjustment control 50 of the respective strut 22 to set the permitted dynamization travel based on an amount specified by one or more of the adjustments in the dynamization schedule 73B which may adjust the axial rigidity of the strut 22 and may permit a collapse and/or expansion of the strut 22 by a (e.g., predetermined) amount. In the implementation of FIGS. 6B and 7, movement of the strut body 30 during the collapse and/or expansion may be limited based on a distance between the retention pin 46 and adjustment control 50. The dynamization travel may be limited to axial movement of the strut body 30 along the strut axis SX. The adjustments may be individually and/or collectively performed to vary the amount of permitted dynamization travel of the respective struts 22 such that the direction of collapse CD of the fixation device 20 may be substantially parallel to the displacement vector and/or substantially perpendicular to the resection plane.

At block 190L, the external fixation device 20 may be removed from the patient anatomy. The external fixation device 20 may be removed from the patient anatomy once the adjustments specified in the dynamization schedule 73B are completed.

The novel planning systems and methods of this disclosure may be utilized to determine an amount of dynamization during bone consolidation, which may occur subsequent to a correction of a deformed bone. The amount of dynamization may be associated with a total dynamization displacement of each strut of the external fixation system (e.g., frame), which may be allocated to a dynamization schedule to achieve a target displacement. The strut may be configurable to set a (e.g., permitted) amount of dynamization travel, which may include a collapse and/or expansion of the strut during dynamization. The disclosed techniques may be utilized to achieve substantially pure axial dynamization along the target (e.g., prescribed) displacement vector, which may be substantially perpendicular to the resection (e.g., osteotomy) plane. Adjustment values may be determined for making substantially pure axial adjustments of the struts of the external fixation device. Shear and torsional forces along the bone interface may be eliminated or otherwise reduced, which may reduce dynamic bone shear forces and a risk of non-unions and malunions and which may promote healing during consolidation of the corrected bone.

Although the different non-limiting embodiments are illustrated as having specific components or steps, the embodiments of this disclosure are not limited to those particular combinations. It is possible to use some of the components or features from any of the non-limiting embodiments in combination with features or components from any of the other non-limiting embodiments.

It should be understood that like reference numerals identify corresponding or similar elements throughout the several drawings. It should further be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure.

1. A system for planning an orthopaedic procedure comprising:
one or more processors coupled to memory, wherein the one or more processors are operable to collectively execute a planning environment, and the planning environment is operable to:
establish a resection plane along a bone model associated with a bone of a patient;
determine a displacement vector associated with compression of the bone adjacent to a resection associated with the resection plane during consolidation, wherein the displacement vector is substantially normal to the resection plane;
place a fixation model relative to the bone model, wherein the fixation model is associated with an external fixation device including a plurality of struts interconnecting first and second supports securable to the bone, and the struts are adjustable to collectively establish a direction of collapse of the external fixation device;
determine a set of strut vectors associated with the respective struts; and
establish a dynamization schedule including adjustments that vary an amount of permitted dynamization travel of one or more of the struts based on the strut vectors such that the direction of collapse is substantially parallel to the displacement vector.

2. The system as recited in claim 1, wherein the permitted dynamization travel includes a collapse and/or expansion of the respective one or more struts.

3. The system as recited in claim 1, wherein the planning environment is operable to:
determine the adjustments based on regression, the regression based on the strut vectors and the displacement vector.

4. The system as recited in claim 3, wherein the regression includes a least-squares regression comprising:
a strut matrix associated with the strut vectors; and
a Gram matrix based on the strut matrix and a Gram matrix determinant, the Gram matrix determinant associated with a distribution of movement of the struts to collectively establish the direction of collapse.

5. The system as recited in claim 4, wherein the least-squares regression comprises:
a pseudo-motion vector that establishes a set of best-fit translation coefficients, wherein the pseudo-motion vector is based on an inversion of the Gram matrix and the displacement vector;
a projection of the pseudo-motion vector onto the strut vectors that establishes dynamization amounts; and
respective amounts of total dynamization displacement for the struts that establish the adjustments in the dynamization schedule, wherein the amounts of total dynamization displacements are based on a magnitude of the displacement vector, a magnitude of the pseudo-motion vector and the dynamization amounts.

6. The system as recited in claim 1, wherein one or more of the adjustments causes an increase in the amount of permitted dynamization travel of one or more of the respective struts.

7. The system as recited in claim 1, wherein one or more of the adjustments causes a decrease in the amount of permitted dynamization travel of one or more of the respective struts.

8. The system as recited in claim 1, wherein the adjustments include different amounts of permitted dynamization travel associated with the struts.

9. The system as recited in claim 1, wherein the strut comprises:
a strut body;
a strut shaft translatably received within an internal cavity of the strut body to set a length of the strut associated with the respective strut vector; and
a dynamization mechanism adjustable to vary the amount of permitted dynamization travel of the strut.

10. The system as recited in claim 9, wherein:
the dynamization mechanism includes a dynamization adjustment control adapted to move relative to a dynamization shaft to either lock or unlock dynamization of the strut in respective locked and unlocked modes;
a position of the dynamization adjustment control adjustable in the unlocked mode to vary the amount of permitted dynamization travel of the respective strut; and
the strut body is moveable relative to the dynamization shaft in the unlocked mode but not in the locked mode.

11. The system as recited in claim 10, wherein:
the dynamization mechanism includes a spring member that opposes axial movement of the strut body relative to the dynamization shaft in the unlocked mode.

12-13. (canceled)

14. The system as recited in claim 1, wherein:
the resection plane is associated with a resection that divides the bone into a first portion attached to the first support and a second portion attached to the second support.

15. The system as recited in claim 1 wherein:
the external fixation device includes a hexapod configuration having six of the struts.

16. A system for planning an orthopaedic procedure comprising:
one or more processors coupled to memory, wherein the one or more processors are operable to collectively execute a planning environment, and the planning environment is operable to:
establish a resection plane along a three-dimensional bone model associated with a bone of a patient, wherein first and second portions of the bone model are on opposite sides of the resection plane;
determine a displacement vector associated with compression of the bone during consolidation, wherein the displacement vector is substantially normal to the resection plane;
place a three-dimensional fixation model about a periphery of the bone model, wherein the fixation model includes a plurality of struts distributed about an axis and interconnecting first and second supports, the first and second supports have positions fixed relative to the respective first and second portions of the bone model, and the struts are adjustable to collectively establish a direction of collapse of the fixation model;
determine a set of strut unit vectors associated with the respective struts; and
determine adjustments that vary an amount of permitted dynamization travel of the respective struts based on the strut unit vectors such that the direction of collapse is substantially parallel to the displacement vector in response to the adjustments.

17. The system as recited in claim 16, wherein the planning environment is operable to:
determine an amount of total dynamization displacement of the respective struts to achieve a target displacement associated with the displacement vector based on regression, and the regression is based on the strut unit vectors and the displacement vector.

18. The system as recited in claim 16, wherein the adjustments have different amounts of permitted dynamization travel associated with the struts.

19. A method of performing an orthopaedic procedure comprising:
establishing a resection plane along a bone model associated with a bone of a patient;
determining a displacement vector associated with compression of the bone during consolidation, the compression along a resection associated with the resection plane, wherein the displacement vector is substantially normal to the resection plane;
placing a fixation model relative to the bone model, wherein the fixation model is associated with an external fixation device and includes a plurality of struts interconnecting first and second supports securable to the bone, and the struts are collectively establish a direction of collapse of the external fixation device;
determining a set of strut unit vectors associated with the respective struts; and
establishing a dynamization schedule including adjustments that vary an amount of permitted dynamization travel of the respective struts based on the strut unit vectors such that the direction of collapse is substantially parallel to the displacement vector.

20. The method as recited in claim 19, wherein the step of establishing the dynamization schedule includes determining the adjustments based on regression, and the regression is based on the strut unit vectors and the displacement vector.

21. The method as recited in claim 20, wherein:
the step of performing the regression comprises:
establishing a strut matrix associated with the strut unit vectors; and
establishing a Gram matrix based on the strut matrix and a Gram matrix determinant, wherein the Gram matrix determinant is associated with a distribution of movement of the struts to collectively establish the direction of collapse; and
the step of performing the regression comprises:
determining a pseudo-motion vector that establishes a set of best-fit translation coefficients, wherein the pseudo-motion vector is based on an inversion of the Gram matrix and the displacement vector; and
determining a projection of the pseudo-motion vector onto the strut unit vectors to determine the adjustments in the dynamization schedule.

22. (canceled)

23. The method as recited in claim 19, wherein the external fixation device is secured to the bone, and further comprising:
adjusting the struts of the external fixation device during consolidation of the bone according to the dynamization schedule; and
moving a dynamization adjustment control of the strut based on an amount specified by one of the adjustments in the dynamization schedule to adjust the amount of permitted dynamization travel of the strut.

24. (canceled)
